(19) United States
(12) Patent Application Publication
(10) Pub. No.: US 2012/0077716 A1
(43) Pub. Date: Mar. 29, 2012

(54) SYSTEM AND METHOD FOR PRODUCING FUNCTIONALLY DISTINCT NUCLEIC ACID LIBRARY ENDS THROUGH USE OF DEOXYINOSINE

(75) Inventors: Brian Christopher Godwin, North Haven, CT (US); Craig Elder Mealmaker, Hamden, CT (US)

(73) Assignee: 454 LIFE SCIENCES CORPORATION, Branford, CT (US)

(21) Appl. No.: 13/233,653

(22) Filed: Sep. 15, 2011

Related U.S. Application Data
(60) Provisional application No. 61/387,512, filed on Sep. 29, 2010.

(51) Int. Cl. C40B 50/06; C07H 21/04

(52) U.S. Cl. 506/26; 536/23.1

(57) ABSTRACT

An embodiment of a nucleic acid adaptor is described that comprises a double stranded nucleic acid element that comprises a plurality of deoxyinosine species positionally located in a spaced relationship from each other on a first strand and base pair to an A, T, or G nucleotide species on a second strand, where a first end of the double stranded nucleic acid element is constructed and arranged to preferentially ligate to each end of a double stranded target nucleic acid molecule.

### CROSS-REFERENCE TO RELATED APPLICATIONS

This application is related to and claims priority from U.S. Provisional Patent Application Ser. No. 61/387,512, titled “System and Method for Producing Functionally Distinct Nucleic Acid Library Fragment Ends Through Use of Deoxyinosine”, filed Sep. 29, 2010, which is hereby incorporated by reference herein in its entirety for all purposes.

### SEQUENCE LISTING

The instant application contains a sequence listing, which has been submitted in ASCII format via EFS-Web and is hereby incorporated by reference in its entirety. The ASCII copy, created on Sep. 7, 2011, is named “549001US.txt” and is 7,578 bytes in size.

### FIELD OF THE INVENTION

The present invention relates to the fields of molecular biology and nucleic acid sequencing instrumentation. More specifically, the invention relates to efficient processing of nucleic acids using methods and unique adaptor elements to produce nucleic acid libraries amenable for sequencing.

### BACKGROUND OF THE INVENTION

There have been a number of advancements in the field of molecular biology that have enabled the development of many technologies that provide great insight into the nature of biological mechanisms. The power of some of these technologies has made great impacts upon scientific discovery and hold great promise for the future. Importantly, some of these technologies are complementary to each other and may be used synergistically to speed the rate at which science gains an understanding of biological systems. It will be appreciated that the field of molecular biology is extremely complex and developers of such technologies may find new uses for previously known mechanisms, but the same developers will build upon new discovery and understanding of biological mechanisms derived through advances in the field of molecular biology.

For instance, there are a number of “nucleic acid sequencing” techniques known in the art that have delivered tremendous contributions to scientific knowledge and hold great promise for future advancements in scientific discovery as well as diagnostic application. Older nucleic acid sequencing techniques include what are commonly known to those of ordinary skill in the art as Sanger type sequencing methods, which employ termination and size separation techniques to identify nucleic acid composition. More recently developed sequencing techniques include techniques such as Sequencing by Hybridization (SBH), Sequencing by Ligation, waveguide, or nanopore techniques. Other powerful sequencing techniques include what are referred to as “sequencing-by-synthesis” techniques (SBS), and include “Pyrosequencing”. SBS techniques are generally employed for determining the identity or nucleic acid composition of one or more molecules in a nucleic acid sample. SBS techniques provide many desirable advantages over previously employed sequencing techniques. For example, embodiments of SBS are enabled to perform high throughput sequencing that generates a large volume of high quality sequence information at a low cost relative to older techniques. A further advantage includes the simultaneous generation of sequence information from multiple template molecules in a massively parallel fashion. In other words, multiple nucleic acid molecules derived from one or more samples are simultaneously sequenced in a single process.

Typical embodiments of SBS comprise the stepwise synthesis of strands of polynucleotide molecules each complementary to a strand from a population of substantially identical template nucleic acid molecules. For example, SBS techniques typically operate by adding a single nucleotide (also referred to as a nucleotide or nucleic acid species) to each nascent polynucleotide molecule in the population where the added nucleotide species is complementary to a nucleotide species of a corresponding template molecule at a particular sequence position. The addition of the nucleic acid species to the nascent molecules typically occurs in parallel for the population at the same sequence position and are detected using a variety of methods known in the art that include, but are not limited to, pyrosequencing that detects liberated pyrophosphate molecules from incorporation events, pH detection techniques that detects liberated hydrogen molecules in response to incorporation events, or fluorescent detection methods such as fluorescent detection techniques employing reversible or “virtual” terminators. Typically, the SBS process is iterative until a complete (i.e. all sequence positions of the target nucleic acid molecule are represented) or desired sequence length complementary to the template is synthesized.

In some embodiments of SBS a number of enzymatic reactions take place in order to produce a detectable signal from each incorporated nucleic acid species. In the pyrosequencing SBS method referred to above, an enzymatic cascade is employed, where each enzyme species in the cascade operates to modify or utilize the product from a previous step. When each nucleotide species is incorporated into the nascent strand, there is a release of an inorganic pyrophosphate molecule (also referred to as PPi) and a hydrogen molecule into the reaction environment. An ATP sulfurylase enzyme is present in the reaction environment and converts PPi to ATP, which in turn is catalyzed by the luciferase enzyme to release a photon of light. Additional enzymes may be used in the cascade to improve the discretion of signals between exposures to different nucleotides species as well as the overall ability to detect signals. Such additional enzymes include, without limitation, one or more of apyrase that degrades unincorporated nucleotide species and ATP, exonuclease that degrades linear nucleic acid molecules, pyrophosphatase (also referred to as PPi-ase) which degrades PPi, or enzymes that inhibit activity of other enzymes. Additional examples of enzymatic improvements for signal discretion are described in U.S. patent application Ser. No. 12/215,455, titled “System and Method For Adaptive Reagent Control in Nucleic Acid Sequencing”, filed Jun. 27, 2008; and U.S. patent application Ser. No. 12/322,284, titled “System and Method for Improved Signal Detection in Nucleic Acid Sequencing”, filed Jan. 29, 2009, each of which is hereby incorporated by reference herein in its entirety for all purposes.

Further, some embodiments of SBS may be performed using instrumentation that automates one or more steps or operation associated with the preparation and/or sequencing methods. Some instruments employ elements, such as plates with wells or other type of microreactor configuration that provide the ability to perform reactions in each of the wells or microreactors simultaneously. Additional examples of SBS techniques as well as systems, consumables, and methods for massively parallel sequencing are described in U.S. Pat. Nos. 6,274,320; 6,258,568; 6,210,891; 7,211,390; 7,244,559; 7,264,929; 7,323,305; 7,335,762; 7,575,865; 7,601,499; and 7,682,816, each of which is hereby incorporated by reference herein in their entireties for all purposes.

It is generally desirable to continually improve technologies such as the sequencing technologies described above to enhance the abilities of scientists to provide insight into biological questions. In preferred embodiments, such improvements are aimed to reduce cost, increase throughput and efficiency, as well as to improve data quality by increasing sensitivity and specificity. Therefore, it is significantly advantageous to continue to develop nucleic acid sequencing technologies, applying the knowledge and understanding of the field of molecular biology to provide more efficient and powerful discovery tools.

Aspects of the invention described herein employ several molecular biology concepts in a new and inventive way to improve the efficiency of processing samples that reduce costs, eliminate steps, and improve data quality.

### SUMMARY OF THE INVENTION

The present invention relates to the determination of the sequence of nucleic acids. More specifically, the invention relates to efficient processing of nucleic acids using methods and unique adaptor elements to produce libraries amenable for sequencing.

An embodiment of a nucleic acid adaptor is described, comprising a double stranded nucleic acid element that comprises a plurality of deoxyinosine species positionally located in a spaced relationship from each other on a first strand and base pair to an A, T, or G nucleotide species on a second strand, where a first end of the double stranded nucleic acid element is constructed and arranged to preferentially ligate to each end of a double stranded target nucleic acid molecule.

Also an embodiment of a kit comprising the nucleic acid adaptor is described.

Further, a method for preparing a nucleic acid library is described that comprises the steps of: ligating a fully complementary double stranded nucleic acid adaptor to each end of a double stranded nucleic acid target molecule to produce a ligated double stranded adaptor-target-adaptor molecule, where the fully complementary double stranded nucleic acid adaptor comprises a plurality of deoxyinosine species positionally located in a spaced relationship from each other on a first adaptor strand that base pair to an A, T, or G nucleotide species on a second adaptor strand; denaturing the double stranded adaptor-target-adaptor molecule to produce two single stranded adaptor-target-adaptor molecules, wherein the single stranded adaptor-target-adaptor molecules comprise a first region from the first adaptor strand at a first end, a middle region from the nucleic acid target molecule, and a second region from the second adaptor strand at a second end; annealing a first primer to the second region of a first strand of the single stranded adaptor-target-adaptor molecules; extending the first primer to produce a second strand complementary to the first strand, wherein the second strand comprises a third region at the second end comprising C nucleotide species base paired to the deoxyinosine species in the first region of the first strand; annealing a second primer to the third region of second strand; and extending the second primer to produce a third strand complementary to the second strand, wherein the second and third strands comprise semi-complementary ends resistant to hybridization to each other.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 160 appears first in FIG. 1). All of these conventions, however, are intended to be typical or illustrative, rather than limiting.

FIG. 1 is a functional block diagram of one embodiment of a sequencing instrument under computer control and a reaction substrate;

FIG. 2 is a simplified graphical representation of the chemical structure of the nucleotide analog Deoxyinosine;

FIG. 3 is a simplified graphical representation of embodiments of adaptor element used in producing functionally distinct ends of a strand nucleic acid targets. FIG. 3 also discloses SEQ ID NOS: 9-11, 10, 12, and 10, respectively, in order of appearance;

FIG. 4 is a simplified graphical representation of one embodiment of the adaptor element of FIG. 3 directionally ligated to a target nucleic acid element and an amplified product produced from the ligated adaptor-target-adaptor complex. FIG. 4 also discloses SEQ ID NOS: 9, 13, 13, 9, 9, 13, 13, 9, 14-16, and 3, respectively, in order of appearance;

FIG. 5 is a simplified graphical representation of one embodiment of a process of polymerase extension of the adaptor-target-adaptor complex resulting in the amplified product of FIG. 4. FIG. 5 also discloses SEQ ID NOS: 5, 4, 4-5, 4-5, 14, 4-5, 14-15, 4, 1, 14-15, 14-15, 3, 14-15, 4, and 3, respectively, in order of appearance; and

FIG. 6 is a simplified graphical representation of sequence data generated from the individual strands of the amplified nucleic acid target of FIGS. 4 and 5.

### DETAILED DESCRIPTION OF THE INVENTION

As will be described in greater detail below, embodiments of the present invention include adaptors capable of producing functionally distinct ends of double stranded nucleic acid molecules. In particular, embodiments of the invention relate to preparation of double stranded nucleic acid libraries amenable for sequencing by “T-A” ligation of sequencing/amplification adaptors containing deoxyinosine species and which are fully double stranded pre-amplification yet only semi-complementary post-amplification.

### a. General

The term “flowgram” generally refers to a graphical representation of sequence data generated by SBS methods, particularly pyrophosphate based sequencing methods (also referred to as “pyrosequencing”) and may be referred to more specifically as a “pyrogram”.

The term “read” or “sequence read” as used herein generally refers to the entire sequence data obtained from a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecules.

The term “flow” as used herein generally refers to a single cycle that is typically part of an iterative process of introduction of fluid solution to a reaction environment comprising a template nucleic acid molecule, where the solution may include a nucleotide species for addition to a nascent molecule or other reagent, such as buffers, wash solutions, or enzymes that may be employed in a sequencing process or to reduce carryover or noise effects from previous flows of nucleotide species.

The term “flow cycle” as used herein generally refers to a sequential series of flows where a fluid comprising a nucleotide species is flowed once during the cycle (i.e. a flow cycle may include a sequential addition in the order of T, A, C, G nucleotide species, although other sequence combinations are also considered part of the definition). Typically, the flow cycle is a repeating cycle having the same sequence of flows from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

The term “test fragment” or “TF” as used herein generally refers to a nucleic acid element of known sequence composition that may be employed for quality control, calibration, or other related purposes.

The term “primer” as used herein generally refers to an oligonucleotide that acts as a point of initiation of DNA synthesis under conditions in which synthesis of a primer extension product complementary to a nucleic acid strand is induced in an appropriate buffer at a suitable temperature. A primer is preferably a single stranded oligodeoxyribonucleotide.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule. “Natural” nucleotide species include, e.g., adenine, guanine, cytosine, uracil, and thymine. Modified versions of the above natural nucleotide species include, without limitation, hypoxanthine, xanthine, 7-methylguanine, 5,6-dihydrouracil, and 5-methylcytosine.

The term “monomer repeat” or “homopolymers” as used herein generally refers to two or more sequence positions comprising the same nucleotide species (i.e. a repeated nucleotide species).

The term “homogeneous extension” as used herein generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products, such as those produced from Polymerase Chain Reaction or Ligase Chain Reaction techniques.

The term “variant” or “allele” as used herein generally refers to one of a plurality of species each encoding a similar sequence composition, but with a degree of distinction from each other. The distinction may include any type of variation known to those of ordinary skill in the related art, that include, but are not limited to, polymorphisms such as single nucleotide polymorphisms (SNPs), insertions or deletions (the combination of insertion/deletion events are also referred to as “indels”), differences in the number of repeated sequences (also referred to as tandem repeats), and structural variations.

The term “allele frequency” or “allelic frequency” as used herein generally refers to the proportion of all variants in a population that is comprised of a particular variant.

The term “key sequence” or “key element” as used herein generally refers to a nucleic acid sequence element (typically of about 4 sequence positions, i.e., TGAC or other combination of nucleotide species) associated with a template nucleic acid molecule in a known location (i.e., typically included in a ligated adaptor element) comprising known sequence composition that is employed as a quality control reference for sequence data generated from template molecules. The sequence data passes the quality control if it includes the known sequence composition associated with a Key element in the correct location.

The term “keypass” or “keypass well” as used herein generally refers to the sequencing of a full length nucleic acid test sequence of known sequence composition (i.e., a “test fragment” or “TF” as referred to above) in a reaction well, where the accuracy of the sequence derived from TF sequence and/or Key sequence associated with the TF or in an adaptor associated with a target nucleic acid is compared to the known sequence composition of the TF and/or Key and used to measure of the accuracy of the sequencing and for quality control. In typical embodiments, a proportion of the total number of wells in a sequencing run will be keypass wells which may, in some embodiments, be regionally distributed.

The term “blunt end” as used herein is interpreted consistently with the understanding of one of ordinary skill in the related art, and generally refers to a linear double stranded nucleic acid molecule having an end that terminates with a pair of complementary nucleotide base species, where a pair of blunt ends are typically compatible for ligation to each other.

The term “sticky end” or “overhang” as used herein is interpreted consistently with the understanding of one of ordinary skill in the related art, and generally refers to a linear double stranded nucleic acid molecule having one or more unpaired nucleotide species at the end of one strand of the molecule, where the unpaired nucleotide species may exist on either strand and include a single base position or a plurality of base positions (also sometimes referred to as “cohesive end”).

The term “SPRI” as used herein is interpreted consistently with the understanding of one of ordinary skill in the related art, and generally refers to the patented technology of “Solid Phase Reversible Immobilization” wherein target nucleic acids are selectively precipitated under specific buffer conditions in the presence of beads, where said beads are often carboxylated and paramagnetic. The precipitated target nucleic acids immobilize to said beads and remain bound until removed by an elution buffer according to the operator's needs (DeAngelis, Margaret M. et al: Solid-Phase Reversible Immobilization for the Isolation of PCR Products. Nucleic Acids Res (1995), Vol. 23:22; 4742-4743, which is hereby incorporated by reference herein in its entirety for all purposes).

The term “carboxylated” as used herein is interpreted consistently with the understanding of one of ordinary skill in the related art, and generally refers to the modification of a material, such as a microparticle, by the addition of at least one carboxyl group. A carboxyl group is either COOH or COO—.

The term “paramagnetic” as used herein is interpreted consistently with the understanding of one of ordinary skill in the related art, and generally refers to the characteristic of a material wherein said material's magnetism occurs only in the presence of an external, applied magnetic field and does not retain any of the magnetization once the external, applied magnetic field is removed.

The term “bead” or “bead substrate” as used herein generally refers to any type of solid phase particle of any convenient size, of irregular or regular shape and which is fabricated from any number of known materials such as cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and other solid phase bead supports known to those of skill in the art.

The term “reaction environment” as used herein generally refers to a volume of space in which a reaction can take place typically where reactants are at least temporarily contained or confined allowing for detection of at least one reaction product. Examples of a reaction environment include but are not limited to cuvettes, tubes, bottles, as well as one or more depressions, wells, or chambers on a planar or non-planar substrate.

The term “virtual terminator” as used herein generally refers to terminators substantially slow reaction kinetics where additional steps may be employed to stop the reaction such as the removal of reactants.

Some exemplary embodiments of systems and methods associated with sample preparation and processing, generation of sequence data, and analysis of sequence data are generally described below, some or all of which are amenable for use with embodiments of the presently described invention. In particular, the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample should be prepared and processed from its raw form into template molecules amenable for high throughput sequencing. The processing methods may vary from application to application, resulting in template molecules comprising various characteristics. For example, in some embodiments of high throughput sequencing, it is preferable to generate template molecules with a sequence or read length that is at least comparable to the length that a particular sequencing method can accurately produce sequence data for. In the present example, the length may include a range of about 25-30 bases, about 50-100 bases, about 200-300 bases, about 350-500 bases, about 500-1000 bases, greater than 1000 bases, or any other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids and may include what is referred to as nebulization or sonication methods. It will, however, be appreciated that other methods of fragmentation, such as digestion using restriction endonucleases, may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements (i.e. such as Key elements or other type of quality control element), unique identifiers (also referred to as a multiplex identifier or “MID”) that encode various associations such as with a sample of origin or patient, or other functional element.

For example, some embodiments of the described invention comprise associating one or more embodiments of an MID element having a known and identifiable sequence composition with a sample, and coupling the embodiments of MID element with template nucleic acid molecules from the associated samples. The MID coupled template nucleic acid molecules from a number of different samples are pooled into a single “Multiplexed” sample or composition that can then be efficiently processed to produce sequence data for each MID coupled template nucleic acid molecule. The sequence data for each template nucleic acid is de-convoluted to identify the sequence composition of coupled MID elements and association with sample of origin identified. In the present example, a multiplexed composition may include representatives from about 384 samples, about 96 samples, about 50 samples, about 20 samples, about 16 samples, about 12 samples, about 10 samples, or other number of samples. Each sample may be associated with a different experimental condition, treatment, species, or individual in a research context. Similarly, each sample may be associated with a different tissue, cell, individual, condition, drug or other treatment in a diagnostic context. Those of ordinary skill in the related art will appreciate that the numbers of samples listed above are provided for exemplary purposes and thus should not be considered limiting.

In preferred embodiments, the sequence composition of each MID element is easily identifiable and resistant to introduced error from sequencing processes. Some embodiments of MID element comprise a unique sequence composition of nucleic acid species that has minimal sequence similarity to a naturally occurring sequence. Alternatively, embodiments of a MID element may include some degree of sequence similarity to naturally occurring sequence.

Also, in preferred embodiments, the position of each MID element is known relative to some feature of the template nucleic acid molecule and/or adaptor elements coupled to the template molecule. Having a known position of each MID is useful for finding the MID element in sequence data and interpretation of the MID sequence composition for possible errors and subsequent association with the sample of origin.

For example, some features useful as anchors for positional relationship to MID elements may include, but are not limited to, the length of the template molecule (i.e. the MID element is known to be so many sequence positions from the 5′ or 3′ end), recognizable sequence markers such as a Key element and/or one or more primer elements positioned adjacent to a MID element. In the present example, the Key and primer elements generally comprise a known sequence composition that typically does not vary from sample to sample in the multiplex composition and may be employed as positional references for searching for the MID element. An analysis algorithm implemented by application 135 may be executed on computer 130 to analyze generated sequence data for each MID coupled template to identify the more easily recognizable Key and/or primer elements, and extrapolate from those positions to identify a sequence region presumed to include the sequence of the MID element. Application 135 may then process the sequence composition of the presumed region and possibly some distance away in the flanking regions to positively identify the MID element and its sequence composition.

Some or all of the described functional elements may be combined into adaptor elements that are coupled to nucleotide sequences in certain processing steps. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In some embodiments, two sets of priming sequence regions (hereafter referred to as priming sequence A, and priming sequence B) may be employed for strand selection, where only single strands having one copy of priming sequence A and one copy of priming sequence B is selected and included as the prepared sample. In alternative embodiments, design characteristics of the adaptor elements eliminate the need for strand selection. The same priming sequence regions may be employed in methods for amplification and immobilization where, for instance, priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; U.S. patent application Ser. No. 12/156,242, titled “System and Method for Identification of Individual Samples from a Multiplex Mixture”, filed May 29, 2008; and U.S. patent application Ser. No. 12/380,139, titled “System and Method for Improved Processing of Nucleic Acids for Production of Sequencable Libraries”, filed Feb. 23, 2009, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR™ methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid, such as a water based fluid suspended or dispersed as droplets (also referred to as a discontinuous phase) within another fluid, such as a hydrophobic fluid (also referred to as a continuous phase) that typically includes some type of oil. Examples of oil that may be employed include, but are not limited to, mineral oils, silicone based oils, or fluorinated oils.

Further, some emulsion embodiments may employ surfactants that act to stabilize the emulsion, which may be particularly useful for specific processing methods such as PCR. Some embodiments of surfactant may include one or more of a silicone or fluorinated surfactant. For example, one or more non-ionic surfactants may be employed that include, but are not limited to, sorbitan monooleate (also referred to as Span™ 80), polyoxyethylenesorbitsan monooleate (also referred to as Tween™ 80), or in some preferred embodiments, dimethicone copolyol (also referred to as Abil® EM90), polysiloxane, polyalkyl polyether copolymer, polyglycerol esters, poloxamers, and PVP/hexadecane copolymers (also referred to as Unimer U-151), or in more preferred embodiments, a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template and amplified copies of the template, amplified copies complementary to the template, or combination thereof. Further, the solid substrate may be enabled for attachment of other type of nucleic acids, reagents, labels, or other molecules of interest.

After emulsion breaking and bead recovery, it may also be desirable in typical embodiments to “enrich” for beads having a successfully amplified population of substantially identical copies of a template nucleic acid molecule immobilized thereon. For example, a process for enriching for “DNA positive” beads may include hybridizing a primer species to a region on the free ends of the immobilized amplified copies, typically found in an adaptor sequence, extending the primer using a polymerase mediated extension reaction, and binding the primer to an enrichment substrate such as a magnetic or Sepharose bead. A selective condition may be applied to the solution comprising the beads, such as a magnetic field or centrifugation, where the enrichment bead is responsive to the selective condition and is separated from the “DNA negative” beads (i.e. no or few immobilized copies).

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. Pat. Nos. 7,638,276; 7,622,280; 7,842,457; 7,927,797; and 8,012,690 and U.S. patent application Ser. No 13/033,240, each of which is hereby incorporated by reference herein in its entirety for all purposes.

Also embodiments sometimes referred to as Ultra-Deep Sequencing, generate target specific amplicons for sequencing may be employed with the presently described invention that include using sets of specific nucleic acid primers to amplify a selected target region or regions from a sample comprising the target nucleic acid. Further, the sample may include a population of nucleic acid molecules that are known or suspected to contain sequence variants comprising sequence composition associated with a research or diagnostic utility where the primers may be employed to amplify and provide insight into the distribution of sequence variants in the sample. For example, a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (first amplicons) is subsequently further amplified individually in separate reaction vessels such as an emulsion based vessel described above. The resulting amplicons (referred to herein as second amplicons), each derived from one member of the first population of amplicons, are sequenced and the collection of sequences are used to determine an allelic frequency of one or more variants present. Importantly, the method does not require previous knowledge of the variants present and can typically identify variants present at <1% frequency in the population of nucleic acid molecules.

Some advantages of the described target specific amplification and sequencing methods include a higher level of sensitivity than previously achieved and are particularly useful for strategies comprising mixed populations of template nucleic acid molecules.

Further, embodiments that employ high throughput sequencing instrumentation, such as for instance embodiments that employ what is referred to as a PicoTiterPlate® array (also sometimes referred to as a PTP™ plate or array) of wells provided by 454 Life Sciences Corporation, the described methods can be employed to generate sequence composition for over 100,000, over 300,000, over 500,000, or over 1,000,000 nucleic acid regions per run or experiment and may depend, at least in part, on user preferences such as lane configurations enabled by the use of gaskets, etc. Also, the described methods provide a sensitivity of detection of low abundance alleles which may represent 1% or less of the allelic variants present in a sample. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005; PCT Patent Application Serial No. US 2008/003424, titled “System and Method for Detection of HIV Drug Resistant Variants”, filed Mar. 14, 2008; and U.S. Pat. No. 7,888,034, titled “System and Method for Detection of HIV Tropism Variants”, filed Jun. 17, 2009, each of which is hereby incorporated by reference herein in its entirety for all purposes.

Further, embodiments of sequencing may include Sanger type techniques, techniques generally referred to as Sequencing by Hybridization (SBH), Sequencing by Ligation (SBL), or Sequencing by Incorporation (SBI) techniques. The sequencing techniques may also include what are referred to as polony sequencing techniques; nanopore, waveguide and other single molecule detection techniques; or reversible terminator techniques. As described above, a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide species to the end of a nascent molecule is substantially the same as that described above for addition to the end of a primer.

As described above, incorporation of the nucleotide species can be detected by a variety of methods known in the art, e.g. by detecting the release of pyrophosphate (PPi) using an enzymatic reaction process to produce light or via detection the release of H⁺ and measurement of pH change (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include, but are not limited to, mass tags and fluorescent or chemiluminescent labels. In typical embodiments, unincorporated nucleotides are removed, for example by washing. Further, in some embodiments, the unincorporated nucleotides may be subjected to enzymatic degradation such as, for instance, degradation using the apyrase or pyrophosphatase enzymes as described in U.S. patent application Ser. No. 12/215,455, titled “System and Method for Adaptive Reagent Control in Nucleic Acid Sequencing”, filed Jun. 27, 2008; and Ser. No. 12/322,284, titled “System and Method for Improved Signal Detection in Nucleic Acid Sequencing”, filed Jan. 29, 2009; each of which is hereby incorporated by reference herein in its entirety for all purposes.

In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 500 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. Pat. No. 7,601,499, titled “Paired end sequencing”; and in U.S. patent application Ser. No. 12/322,119, titled “Paired end sequencing”, filed Jan. 28, 2009, each of which is hereby incorporated by reference herein in its entirety for all purposes.

Some examples of SBS apparatus may implement some or all of the methods described above and may include one or more of a detection device such as a charge coupled device (i.e., CCD camera) or confocal type architecture for optical detection, Ion-Sensitive Field Effect Transistor (also referred to as “ISFET”) or Chemical-Sensitive Field Effect Transistor (also referred to as “ChemFET”) for architectures for ion or chemical detection, a microfluidics chamber or flow cell, a reaction substrate, and/or a pump and flow valves. Taking the example of pyrophosphate-based sequencing, some embodiments of an apparatus may employ a chemiluminescent detection strategy that produces an inherently low level of background noise.

In some embodiments, the reaction substrate for sequencing may include a planar substrate, such as a slide type substrate, a semiconductor chip comprising well type structures with ISFET detection elements contained therein, or waveguide type reaction substrate that in some embodiments may comprise well type structures. Further, the reaction substrate may include what is referred to as a PTP™ array available from 454 Life Sciences Corporation, as described above, formed from a fiber optic faceplate that is acid-etched to yield hundreds of thousands or more of very small wells each enabled to hold a population of substantially identical template molecules (i.e., some preferred embodiments comprise about 3.3 million wells on a 70×75 mm PTP™ array at a 35 μm well to well pitch). In some embodiments, each population of substantially identical template molecule may be disposed upon a solid substrate, such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. An example of reaction substrates comprising characteristics for improved signal recognition is described in U.S. Pat. No. 7,682,816, titled “THIN-FILM COATED MICROWELL ARRAYS AND METHODS OF MAKING SAME”, filed Aug. 30, 2005, which is hereby incorporated by reference herein in its entirety for all purposes. Further examples of apparatus and methods for performing SBS type sequencing and pyrophosphate sequencing are described in U.S. Pat. Nos. 7,323,305 and 7,575,865, both of which are incorporated by reference above.

In addition, systems and methods may be employed that automate one or more sample preparation processes, such as the emPCR™ process described above. For example, automated systems may be employed to provide an efficient solution for generating an emulsion for emPCR processing, performing PCR Thermocycling operations, and enriching for successfully prepared populations of nucleic acid molecules for sequencing. Examples of automated sample preparation systems are described in U.S. Pat. No. 7,927,797; and U.S. patent application Ser. No 13/045,210, each of which is hereby incorporated by reference herein in its entirety for all purposes.

Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

An exemplary embodiment of a computer system for use with the presently described invention may include any type of computer platform such as a workstation, a personal computer, a server, or any other present or future computer. It will, however, be appreciated by one of ordinary skill in the art that the aforementioned computer platforms as described herein are specifically configured to perform the specialized operations of the described invention and are not considered general purpose computers. Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provides one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Celeron®, Core™, or Pentium® processor made by Intel Corporation, a SPARC® processor made by Sun Microsystems, an Athlon™, Sempron™, Phenom™, or Opteron™ processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows®-type operating system (such as Windows® XP, Windows Vista®, or Windows®_{—}7) from the Microsoft Corporation; the Mac OS X operating system from Apple Computer Corp. (such as Mac OS X v10.6 “Snow Leopard” operating systems); a Unix® or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium, such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also, a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example, some commonly employed web browsers include Microsoft® Internet Explorer 8 available from Microsoft Corporation, Mozilla Firefox® 3.6 from the Mozilla Corporation, Safari 4 from Apple Computer Corp., Google Chrome from the Google™ Corporation, or other type of web browser currently known in the art or to be developed in the future. Also, in the same or other embodiments an internet client may include, or could be an element of, specialized software applications enabled to access remote information via a network such as a data processing application for biological applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

### b. Embodiments of the Presently Described Invention

As described above, embodiments of the described invention are directed to improved systems, methods, and kits comprising a double stranded adaptor embodiment for producing libraries of sequencable single stranded templates having functionally distinct ends through the use of deoxyinosine species (also sometimes abbreviated and referred to as dI).

Embodiments of the presently described invention comprise deoxyinosine, a universal nucleotide species that base pairs with varying bond strength to the other naturally occurring nucleotides (Adenine, Guanine, Cytosine and Thymine), incorporated in a single strand of a double stranded adaptor embodiment that is typically ligated to each end of a double stranded template nucleic acid molecule to be amplified. In the described embodiments, the dI species on a “first” strand may be paired with either an “A”, “T”, or “G” nucleotide species on the complementary “second” strand imparting full complementarity of both strands of the adaptor embodiment, wherein amplification of the adaptor embodiment results in a double stranded product having semi-complementarity of the strands. Typical processes of nucleic acid amplification, such as PCR processes, includes a polymerase enzyme that typically recognizes dI species in the “first” strand as a template for “C” nucleotide species incorporation into the “second” complementary extended strand. When the “second” complementary strand is used as a template in a subsequent round of PCR thermocycling, the “C” nucleotide species on the “second” complementary strand is a template for incorporation of a “G” nucleotide species into a copy of a “modified first” strand. Subsequent rounds of PCR thermocycling using the “second” complementary and the “modified first” strands as templates produce products that are complementary copies without further engineered modification of composition (there may be some unexpected modification from PCR errors at a rate as would be typically expected for the particular polymerase species).

As will be described in greater detail below, the scheme employed in the design of adaptor embodiments incorporating dI species allow for creation of amplified products comprising individual strands with end regions derived from the amplified adaptor embodiments, each end having distinct nucleic acid species composition from the other that enables efficient downstream processing where each strand of the amplified nucleic acid template molecules are amenable for sequencing applications.

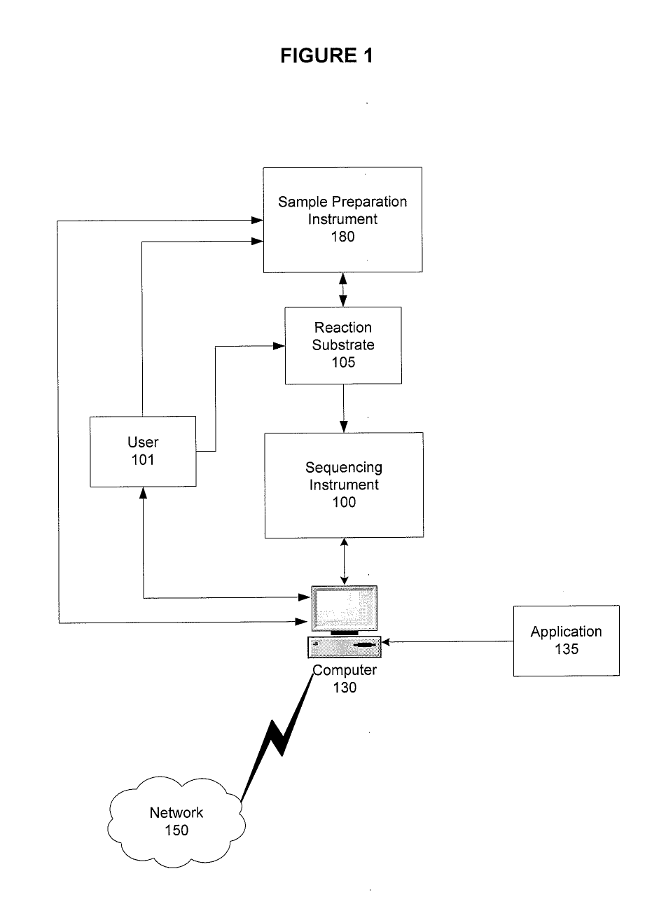

In a typical sequencing embodiment, one or more instrument elements may be employed that automate one or more process steps. For example, embodiments of a sequencing method may be executed using instrumentation to automate and carry out some or all process steps. FIG. 1 provides an illustrative example of sequencing instrument 100 that for sequencing processes requiring capture of optical signals typically comprise an optic subsystem and a fluidic subsystem for execution of sequencing reactions and data capture that occur on reaction substrate 105. It will, however, be appreciated that for sequencing processes requiring other modes of data capture (i.e. pH, temperature, electrochemical, etc.), a subsystem for the mode of data capture may be employed which are known to those of ordinary skill in the related art. For instance, a sample of template molecules may be loaded onto reaction substrate 105 by user 101 or some automated embodiment, then sequenced in a massively parallel manner using sequencing instrument 100 to produce sequence data representing the sequence composition of each template molecule. Importantly, user 101 may include any such user that includes, but is not limited to, an independent researcher, technician, clinician, university, or corporate entity.

In some embodiments, samples may be optionally prepared for sequencing in an automated or partially automated fashion using sample preparation instrument 180 configured to perform some or all of the necessary preparation for sequencing using instrument 100. Examples of sample preparation instruments may include robotic platforms such as those available from Hamilton Robotics, Beckman Coulter, or Caliper Life Sciences. Further, as illustrated in FIG. 1, sequencing instrument 100 may be operatively linked to one or more external computer components, such as computer 130 that may, for instance, execute system software or firmware, such as application 135 that may provide instructional control of one or more of the instruments, such as sequencing instrument 100 or sample preparation instrument 180, and/or data analysis functions. Computer 130 may be additionally operatively connected to other computers or servers via network 150 that may enable remote operation of instrument systems and the export of large amounts of data to systems capable of storage and processing. In the present example, sequencing instrument 100 and/or computer 130 may include some or all of the components and characteristics of the embodiments generally described above.

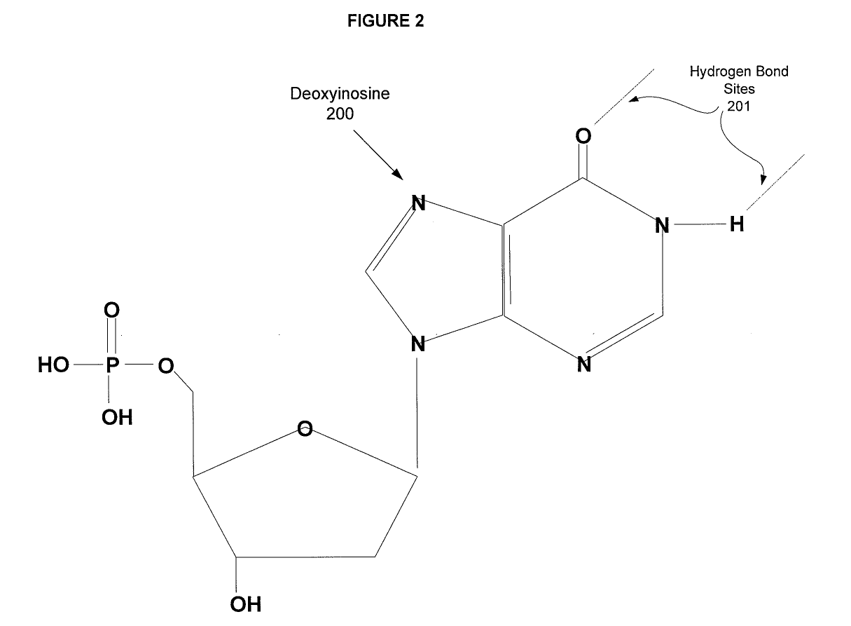

The present invention owes its utility to the nucleotide analog deoxysinosine (dI). As illustrated in the example of FIG. 2, deoxyinosine 200 shares many of the structural similarities to the purine nucleotides Adenosine and Guanosine yet pairs universally with all four nucleotide species with varying bond strengths. It has two available sites for hydrogen bond formation with the other naturally occurring bases, illustrated as hydrogen bond sites 201, and is thus suitable to incorporate into oligonucleotide molecule designs for a multiplicity of uses, including but not limited to creation of adaptors for use in library preparation for sequencing applications. Incorporation of dI species into a single adaptor species embodiment that are subject to PCR based amplification results in the incorporation of a “C” nucleotide species into a complementary second strand at the same sequence position in the first strand as the dI species that in subsequent iterations of thermocycling are used as a template for incorporation of a “G” nucleotide species into a copy of the original first strand with modified composition. Embodiments of the presently described invention are different from previous adaptor embodiment designs because of the unique use of dI species, which produce functionally distinct ends on the amplified population nucleic acid template molecules due to the universal base-pairing and amplification characteristics of dI species.

In the presently described embodiments, the adaptor of the invention comprises several component elements that confer desirable characteristics that are particularly advantageous for use in particular processing steps. The advantages conferred by these component elements enable substantial improvements over processing target molecules operatively coupled to previous adaptor embodiments. For example, processing methods using previous adaptor embodiments are described in U.S. patent application Ser. No. 10/767,894, incorporated by reference above employ two distinct adaptor species (referred to as Adaptor A and Adaptor B) that are randomly ligated to the ends of each target nucleic acid molecule. In the present example, the individual characteristics of the A and B adaptor species make it necessary that each adapted target molecule employed in a sequencing reaction include both an A and B adaptor (i.e. one of each species ligated to an end of the target, represented as A/B adaptor combination). Due to the random nature of the ligation step (i.e. production of A/A, A/B and B/B adapted molecules), subsequent processing steps are necessary to insure that only molecules with an A/B adaptor combination are selected (i.e. strand selection).

In an alternative example, another previous adaptor embodiment is described in U.S. patent application Ser. No. 12/380,139, incorporated by reference above, that employs a single adaptor species that comprises a region of complementarity and a second non-complementary region prior to an amplification step to promote directional ligation of the adaptor species to the target molecule.

The presently described invention provides a substantial improvement over processing with the combination of A/B adaptor species because there is only a single adaptor species that performs the same functions as the A/B adaptor species combination without the strand selection steps required by the A/B adaptor strategy as well as additional advantages that will be illustrated further below. One important characteristic possessed by the adaptor of the present invention is that it has what will be referred to herein as “directional” characteristics and strand specific elements that enable the adaptor to ligate to each end of a linear nucleic acid target molecules in a desired orientation. Further, this directionality when amplified imparts single stranded characteristics to each strand resulting in each strand having the equivalent to an A sequence composition at one end and a B sequence composition at the other without a tedious strand selection procedure. For example, the directional characteristic of the adaptor species of the invention is derived, at least in part, on the directional nature and base pairing relationship of the individual strands of the adaptor molecule. The proper orientation of the adaptor at each end of the target molecule appropriately positions the specific elements of each strand of the adaptor for optimal use in subsequent process steps such as, for instance, amplification and/or sequencing steps.

The described invention also provides a marked improvement in adaptor stability with the inclusion of a plurality of deoxyinosine species. Due to its nature, dI is a universal base that can base pair with all four nucleotide species (A, G, C, & T). It is therefore easily appreciated by one of ordinary skill in the art that use of dI species in adaptor design is of great utility. In the presently described invention, dI species are incorporated into the adaptor embodiment at specific sequence positions which pairs with either an “A”, “T” or “G” nucleotide species and produces a fully complementary double stranded adaptor which confers stability to the adaptor. This design is superior to older embodiments due to the adaptor's complete complementarity and increased stability prior to amplification. Having a completely double stranded adaptor provides greater thermodynamic stability contributed largely by each neighboring base's stabilizing effects through uninterrupted vertical stacking within the duplex helical structure. This is appreciably different from some adaptor embodiments, which have non-complementary segments that disrupt the advantageous base-stacking and thereby decrease the adaptor's stability in comparison.

Another advantage of the adaptor embodiments of the present invention over the previously described A/B adaptor embodiments includes the use of both strands of the adapted target molecule in subsequent steps, as opposed to the production of only a single useable strand from each double stranded adapted target molecule. As described above, the single adaptor species of the presently described invention eliminates the need for strand selection steps required by the A/B adaptor embodiments and produces two sequencable templates from each adapted double stranded molecule.

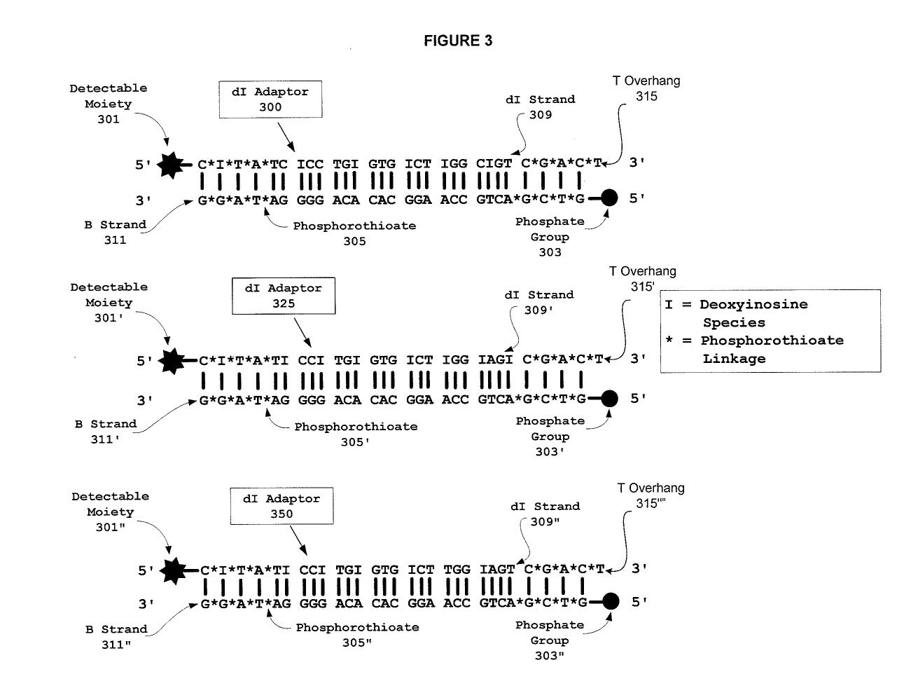

FIG. 3 provides an illustrative example of three embodiments of dI adaptors 300, 325, and 350 each comprise a fully complementary double stranded nucleic acid molecule suitable for ligation to the ends of a target molecule to be amplified and sequenced which could be fragmented genomic DNA, an amplicon, or other type of nucleic acid molecule suitable for sequencing. More specifically FIG. 3 illustrates the fully complementary relationship of individual dI strands 309, 309′, 309″ with B strands 311, 311′ or 311″ respectively each of which follow the Watson-Crick base pairing rules based upon the sequence composition of each strand.

In the described embodiments, it is generally desirable that the dI species are positioned no closer than 6 base positions from the end of the strand which ligates to a target nucleic acid, and in the same or alternative embodiments it may also be desirable that each of the dI species are at least 4 sequences positions away from each other to prevent re-annealing, and further a regular spacing of 4 or 5 sequence positions is desirable. While it is useful to follow these guidelines for adaptor design comprising dI species, adaptor sequence composition can be modified for various applications specific to each user's needs, such as amplification or sequencing primer composition, and it will be appreciated by those of ordinary skill in the art that the examples of dI adaptor embodiment provided herein should not be considered limiting and that the number of dI species and their sequence positions as well as total length of the strands of the adaptors may include a variety of different possible combinations.

FIG. 3 provides exemplary adaptor embodiments 300, 325 and 350 comprising dI species in a single strand that vary in number and sequence position relative to each other and each comprise a nucleotide composition that fully hybridize to form a double stranded adaptor. In the example of dI adaptor 300, the embodiments of dI species are associated with dI strand 309, however it will be appreciated that embodiments of dI species may be associated with B strand 311, or some combination of strands 309 and 311. Also in the present example, the composition and/or length of strands 309; 309′; 309″; or 311; 311′; 311″ may in some cases be dependent upon one or more sequence elements or components encompassed within such primer sequences, quality control elements, unique identifier elements, addition or deletion of dI species or other sequence element known in the art, or some combination thereof.

Also illustrated in FIG. 3 is B strand 311 comprising a phosphate 303 at the 5′ terminus that contributes to the “directionality” of dI adaptor 300 where phosphate 303 promotes directional ligation of the 5′ end of strand 311 in dI adaptor 300 to the 3′ end of each strand of a double stranded nucleic acid target molecule. More specifically, phosphate 303 at the 5′ end of B strand 311 is beneficial for ligation to the 3′ hydroxyl termini of each strand at each end of a nucleic acid target molecule. Those of ordinary skill in the related art will appreciate that an embodiment of adaptor 300 ligates to each end of a nucleic acid target molecule at the end where phosphate 303 is present and thus each strand of the completely ligated adaptor-target-adaptor will include a preferred sequence composition at each adapted end. Further, in the example presented in FIG. 3, the 3′ terminus of dI strand 309 comprises a single “T” nucleotide overhang in dI adaptor 300, illustrated as T overhang 315. T overhang 315 on dI adaptor 300 further promotes the directional ligation described above via modification of nucleic acid target molecules to include a single A species overhang on the 3′ terminus of each strand on the nucleic acid target molecule, thus creating a “T-A” base pairing relationship between adaptor 300 and each end of the nucleic acid target molecule. This is typically accomplished by a polymerase extension reaction known to those of ordinary skill in the art, which occurs during the process of end polishing of the target fragment. It will also be appreciated that the example described above is for illustration purposes and that the same applies to adaptors 325 and 350 as well as other adaptor embodiments comprising the described dI species strategy.

Also illustrated in FIG. 3 are phosphorothioate 305, 305′, and 305″ nucleotide species in the sequence composition. Those of ordinary skill in the related art will appreciate that “phosphorothioates” are analogues of nucleotide species that comprise a sulfur molecule in place of an oxygen molecule as one of the non-bridging ligands bonded to phosphorus. In embodiments of dI adaptor 300, 325, or 350, the incorporation of one or more embodiments of phosphorothioate, one example of which is indicated by phosphorothioate 305, into the sequence composition confers resistance to exonuclease digestion as well as providing improvement to ligation efficiency. In some embodiments, one or more embodiments of phosphorothioate 305 on dI strand 309 is positionally located in a complementary sequence position to one or more embodiments of phosphorothioate 305 on B strand 311. Also, in the same or alternative embodiments there are at least 4 embodiments of phosphorothioate 305 at each end of strands 309 and 311 interspaced by a single embodiment of a natural nucleotide species and having an embodiment of natural nucleotide species at the end position of each strand.

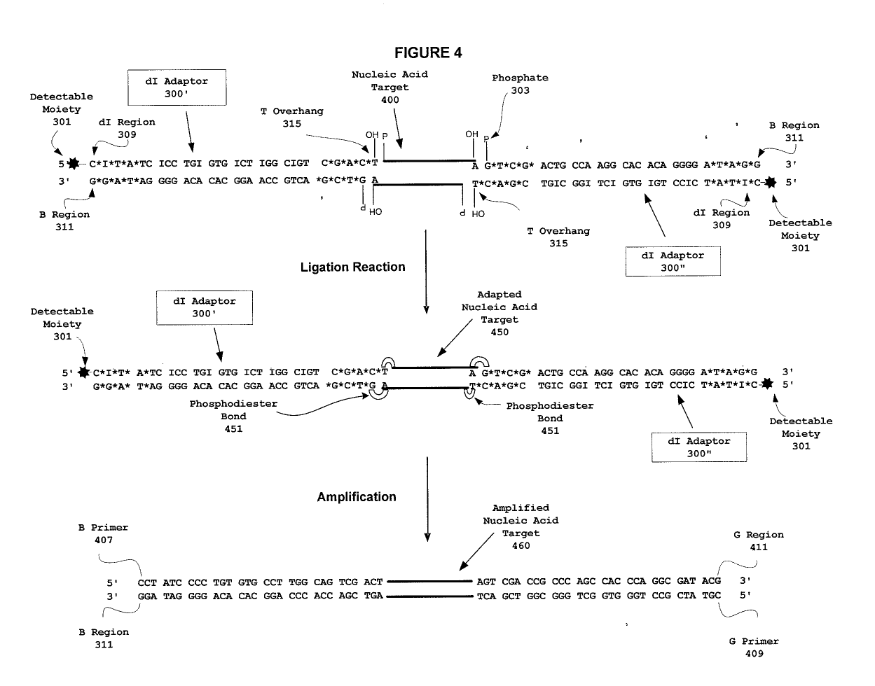

Some embodiments of dI adaptor 300, 325 or 350 may additionally include detectable moiety 301, 301′, or 301″ that enables direct quantification of the number of nucleic acid molecules in a volume rather than employing quantification methods, such as measurements of total mass of nucleic acid molecules and an estimation of the average size of the molecules. Embodiments of detectable moiety 301 may include a fluorescent moiety, enzymatic conjugates (i.e. alkaline phosphatase or horseradish peroxidase), or other type of detectable moiety known to those of ordinary skill. In the described embodiments, detectable moiety 301 is positionally located at the 5′ terminus of dI strand 309 that also contributes to the inhibition of ligation of said end with other molecules. In some embodiments detectable moiety 301 may include a fluorescent moiety that allows for easy, efficient, and accurate quantitation of molecule numbers via detection of light emitted from the attached moieties in a volume of fluid. The amount of detected light may be compared to a standard measure of known association of light to the number of moieties to determine the number of molecules associated. For example, each fluorescent moiety emits a photon of light in response to an absorbed photon of light in the moieties excitation range (also referred to as the absorption range), where the emitted photon is at a longer wavelength than the wavelength of the excitation photon (generally referred to as a “Stokes Shift”). Thus, the intensity of light emitted from a pool of fluorescent moieties in response to a known intensity of excitation light is based, at least in part, upon the number of fluorescent moieties in the pool. In the present example, detectable moiety 301 comprises a single fluorescent moiety associated with each embodiment of dI adaptors 300, 325, or 350, so that each embodiment of adapted nucleic acid target 450 of FIG. 4 comprises two embodiments of detectable moiety 301. Therefore, there is a direct association of the number of fluorescent moieties to the number of adapted nucleic acid molecules in a sample that is easily measurable using standard excitation sources (i.e. laser, LED, UV, or incandescent sources) and detection devices (i.e. Fluorometer, CCD, or confocal detection architectures) known in the art. The species of fluorescent moiety may include, but is not limited to Cy3, Cy5, carboxyfluorescein (FAM), Alexafluor, Rhodamine green, Texas Red, R-Phycoerytherin, semiconductor nanocrystals (also referred to as “Quantum Dots”), or other fluorescent species known in the art.

FIG. 4 provides an illustrative example of embodiments of dI adaptor 300 (indicated as adaptor 300′ and adaptor 300″) arranged for coupling to each end of nucleic acid target 400 via directional ligation. General description of preparing nucleic acid target molecules that includes methods for fragmentation, blunt end polishing, ligation methods (including associated methods, such as “nick fill-in” reactions), and other related processing steps are described in U.S. patent application Ser. Nos. 10/767,894, and 12/380,139, incorporated by reference above.

As described above, it may be advantageous in some embodiments to employ “sticky ends” to promote directional ligation of dI adaptor 300 to nucleic acid target 400. Some of the advantages of using sticky end ligation also include inhibition of target concatemer formation, inhibition of adaptor dimer formation, and inhibition of the circularization of target molecules. In some embodiments, an overhang comprising a single base position on the end of each nucleic acid molecule to be joined is sufficient for providing the various advantages listed above, however it will be appreciated that longer overhangs may also be employed. In the same or alternative embodiments, the overhangs may be reliably created using methods known in the art. For example, genomic input DNA may be used to create nucleic acid targets, such as that described by Nucleic Acid Target 400, through fragmention by any of the methods known in the art and as described in U.S. patent application Ser. No. 10/767,894 incorporated by reference above, and the ends of the nucleic acid fragments may be polished to remove overhangs where the sequence composition may be unknown. Next, the addition of a single base overhang comprising an A nucleotide species to the 3′ ends of nucleic acid target 400 is performed using various methods. One such method uses the “extendase” properties of Taq polymerase. In the present example, the A species extension may be achieved within the fragment end polishing reaction buffer that includes T4 Polymerase and T4 Polynucleotide Kinase (hereafter referred to as PNK) at a temperature of 25° C. for 20 minutes to the T4 polymerase and PNK activity. Next, the temperature is set to 72° C. for 20 minutes for the incorporation of the A nucleotide species and inactivation of the T4 polymerase and PNK. The reactions may also be cleaned up using SPRI technology or purification columns.

In addition, those of ordinary skill in the related art will appreciate that nucleic acid target 400 may be “phosphorylated” at the 5′ ends of individual strands to improve ligation efficiency, as illustrated in FIG. 4 as phosphate 403. In the example illustrated in FIG. 4, the 3′ terminal end of dI strand 309 comprises T overhang 315 that aligns to the A species extended from the 3′ ends of nucleic acid target 400. The 5′ phosphate 403 aligns with a 3′ hydroxyl group at the ends of the strands of nucleic acid target 400 and are ligated.

Also in the example of FIG. 4, T overhang 315 on dI strand 309 at the 3′ terminus will preferentially ligate to embodiments of nucleic acid target 400 that have 3′ terminal A species overhang, such that ligation is directed to those sites and adaptor:adaptor ligation is significantly decreased. Thus, the structural characteristics of each end of dI adaptor 300 that include the positions of phosphate 303 and the T overhang 315 provide directionality to dI adaptor 300 with respect to ligation of target nucleic acid molecules containing the proper 3′ terminal A species overhangs. The result of this directional ligation is that adaptors 300′ and 300″ are in an “inverted” relationship relative to each other, forming adapted nucleic acid target 450.

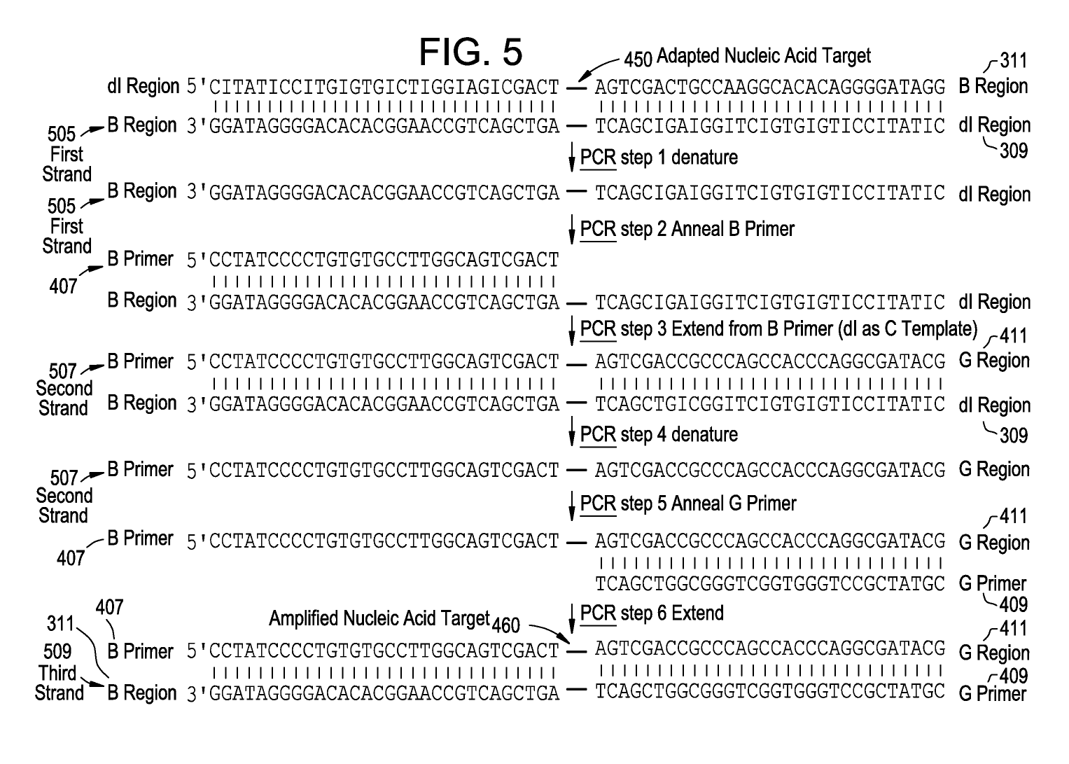

FIG. 4 also illustrates the result of amplifying adapted nucleic acid target 450 using the polymerase based PCR amplification technique described above to produce amplified nucleic acid target 460. FIG. 5 provides an illustrative example of the amplification process described herein with more specific detail of the steps and products produced at each step.

The process illustrated in FIG. 5 begins with adapted nucleic acid target 450 that comprises two strands each having an embodiment of B region 311 and dI region 309. In the first step of PCR, the strands are denatured and treated independently, illustrated as first strand 505. In subsequent PCR cycling, B primer 407 is annealed to B region 311 and extended to produce second strand 507 that comprises G region 411 synthesized using dI region 309 as a template and incorporating C nucleotide species at positions where the dI species is the template. Next, first strand 505 and second strand 507 are denatured and G primer 409 annealed to G region 411 of second strand 507. G primer 409 is then extended to produce third strand 509 that while annealed to second strand 507 makes up amplified nucleic acid target 460. One of ordinary skill in the related art will appreciate that after subsequent rounds of amplification, the predominant species of amplification product comprises amplified nucleic acid target 460 and that some elements of adaptor 300 such as detectable moiety 301 and phosphorothioate 305 are typically lost. It will further be appreciated that both strands 507 and 509 of amplified target nucleic acid 460 are amenable for sequencing, typically by increasing the copy numbers individually using a subsequent clonal amplification process and sequencing the clonally amplified populations.

In some embodiments, the amplification strategy described to produce amplified nucleic acid target 460 could be implemented in a tube, well plate, cuvette, etc., where target 460 could then be subsequently introduced into a clonal amplification system such as the emulsion based strategy for creating clonally amplified populations described above. However, a more efficient strategy is to perform the amplification in the emulsion to produce a clonally amplified population bound to beads. In the described strategy, the individual strands of adapted target nucleic acid 450 may be separated and the single strands introduced into the emulsion droplets. For example, in some emPCR embodiments an amplification primer species is immobilized upon a bead support and a second primer species is in a reaction solution (i.e. in solution phase) both encapsulated within an aqueous droplet which compartmentalizes the reaction environment. In the present example, the immobilized primer species is B primer 407 and the solution phase primer is G primer 409, however those of ordinary skill will appreciate that alternative combinations are also possible.

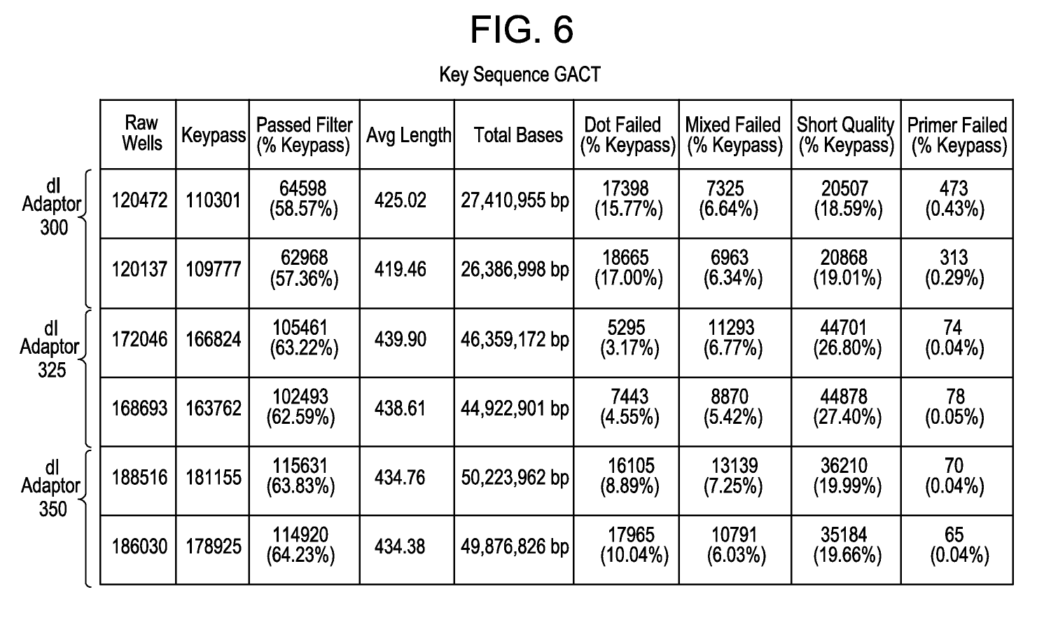

As described above, amplified nucleic acid target 460 comprises a measure of semi-complementarity between B primer region 407 and G region 411 at the ends of strand 507, and a measure of semi-complementarity between B region 311 and G primer 409 at the ends of strand 509 due to the incorporation and conversion effects of the dI species described above. The semi-complementary nature inhibits the formation of secondary structure by self-hybridization of the end regions of single stranded nucleic acid strands 507 and 509 within an amplification reaction, which would decrease efficiencies of the amplification reactions. The semi-complementary nature of the ends also provides sufficient sequence distinctiveness to enable their use as distinct priming sites that provides the proper distinct functional utility necessary for amplification and sequencing processes. The term “semi-complementary” as used herein generally refers to the complementary nature of nucleotide species at sequence positions within the molecule, where a first region comprises a sequence composition between strands that is complementary and a second region that comprises a non or partially-complementary sequence composition depending upon the location and number of dI species. It will be appreciated that the region derived from the adaptor comprising the fully complementary sequence composition is typically located at the end of the adaptor that ligates to the target nucleic acid up to the position of the first dI species, which in the case of adaptor 300 is at least 6 sequence positions. For example, semi-complementarity between the end regions of strands 507 and 509 is present after the embodiments of adapted nucleic acid target 450 have gone through amplification steps such as those described in FIG. 5. In the present example, it is possible that some degree of secondary structure could form within the unknown sequence composition of nucleic acid target 400, yet it will be appreciated by one of ordinary skill in the art that DNA from any source can be composed of varying GC content, which could provide for regions of self-hybridization within molecules. An important consideration that must be appreciated in the present invention is the utility and advantage gained through the use of dI species in adaptor design and how distinct ends of single stranded can be created from a single double stranded adaptor embodiment to produce nucleic acid template molecules amenable for sequencing. FIG. 6 provides an example of data obtained from sequencing runs using each of adaptors 300, 325, and 350. Those of ordinary skill in the art will appreciate that the data of FIG. 6 comprises metrics that demonstrates that each of adaptors 300, 325, and 350 performed very well when used as adaptors as described herein. For example, the average read length for adaptors 300, 325, and 350 were all greater than 400 bases with low rates of primer failure, especially for adaptors 325 and 350.

The presently described invention also includes embodiments of a kit comprising adaptors of the described invention and one or more reagents, enzymes, or other consumable material useful for creating a library of nucleic acid template molecule amenable for sequencing.

### EXAMPLES

### Example 1

### Nucleic Acid Preparation and Fluorescent Quantification

DNA was fragmented by nebulization at 30 psi in a vented nebulizer for 1 minute, then eluted in 16 μl of elution buffer on a MinElute column (Qiagen). The eluted DNA was subjected to polishing and end-repair under conditions of 25° C. for 20 minutes, followed by 72° C. for 20 minutes, and held at 4° C. The polishing and end repair reaction was carried out in 16 μl of the fragmented sample in elution buffer, to which 2.5 μl of Polishing Buffer (454 kit), 2.5 μl ATP (454 kit), 1 μl dNTP (454 kit), 1 μl T4 polynucleotide kinase (454 kit), 1 μl T4 DNA polymerase (454 kit), and 1 μl Taq polymerase (454 kit) were added. A ligation reaction was then carried out, wherein 25 μl of polished/end-repaired sample was incubated with 1 μl of a dI adaptor and 1 μl of ligase for 10 minutes at 25° C. At the conclusion of the ligation reaction, the sample was held at 4° C. Thereafter, the sample was subjected to SPRI size exclusion chromatography to remove adaptor dimers. SPRI beads (125 μl resuspended in 500 μl of Sizing Solution; 454 kit). To 50 μl adapted library fragments (the 27 μl was brought up to 50 μl with 23 μl of 1X TE buffer), 500 μl of SPRI beads were added and rotated at room temperature for 5 minutes. The beads were then subjected to a magnet, the supernatant removed, and the beads were subsequently resuspended in 50 μl 1X TE buffer and 500 μl of sizing solution. The beads were then rotated at room temperature for another 5 minutes, then subjected to the magnet and supernatant removal. The beads were then washed twice with 70% ethanol while beads were still bound to the magnet. The second 70% ethanol wash was removed and the bead pellet allowed to air dry for two minutes. The pellet was resuspended in 50 μl of 1X TE buffer, bound to the magnet, and the supernatant removed and transferred to a separate 1.7 mL Eppendorf tube. The product was quantified using a blue filter on a TBS-380 fluorometer, using a previously quantified FAM oligonucleotide as a standard. The DNA sample was then heat denatured to separate the strands, resulting in a sample containing single stranded DNA product.

### Example 2

### Deoxyinosine Incorporation and Comparison of Binding Energy Between Pre- and Post-Amplification Adaptor Composition

Adaptors were designed with deoxyinosine nucleotides specifically to generate sequencable nucleic acid target molecules. A comparison of the relative binding energy of the amplified products shows the utility and superiority over the previous semi-complementary adaptor designs.

dI adaptor 300 has the following characteristics: 1) Pre-amplification the binding energy is a ΔG of −51.56 kcal/mole; 2) Post-amplification the resulting binding energy is a ΔG of −11.04 kcal/mole.

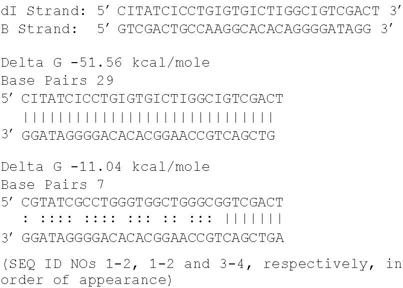

dI adaptor 325 has the following characteristics: 1) Pre-amplification the binding energy is a ΔG of −49.49 kcal/mole; 2) Post-amplification the resulting binding energy is a ΔG of −8.13 kcal/mole.

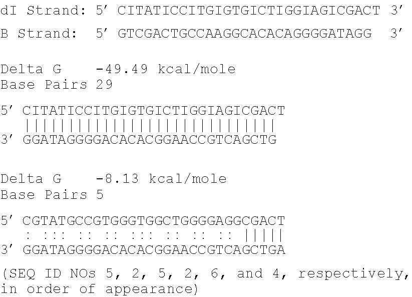

dI adaptor 350 has the following characteristics: 1) Pre-amplification the binding energy is a ΔG of −49.49 kcal/mole; 2) Post-amplification the resulting binding energy is a ΔG of −12.64 kcal/mole.

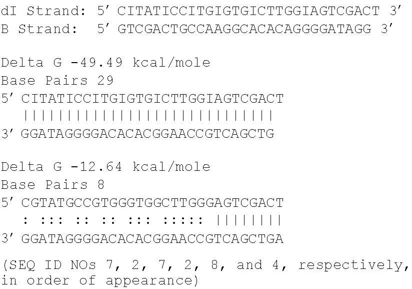

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

What is claimed is:

1. A nucleic acid adaptor, comprising:
a double stranded nucleic acid element that comprises a plurality of deoxyinosine species positionally located in a spaced relationship from each other on a first strand and base pair to an A, T, or G nucleotide species on a second strand, wherein a first end of the double stranded nucleic acid element is constructed and arranged to preferentially ligate to each end of a double stranded target nucleic acid molecule.

2. The nucleic acid adaptor of claim 1, wherein:
the first strand comprises a 3′ single base overhang and a second strand comprises a 5′ phosphate group that preferentially ligates the first end of the double stranded nucleic acid element to each end of the double stranded target nucleic acid molecule.

3. The nucleic acid adaptor of claim 1, wherein:
the first strand comprises a detectable moiety at the 5′ terminus.

4. The nucleic acid adaptor of claim 1, wherein:
the second strand comprises a detectable moiety at the 3′ terminus.

5. The nucleic acid adaptor of claim 1, wherein:
the deoxyinosine species are spaced 4 or more sequence positions away from each other.

6. The nucleic acid adaptor of claim 1, wherein:
the deoxyinosine species are spaced no closer than 6 sequence positions from an end of the double stranded nucleic acid element arranged to ligate to a double stranded target nucleic acid molecule.

7. The nucleic acid adaptor of claim 1, wherein:
the first and the second strands comprise a plurality of phophorothioates at each end.

8. The nucleic acid adaptor of claim 7, wherein:
one or more of the phophorothioates at each end of the first strand are positionally located in a complementary relationship to one or more of the phosphorothioates at each end of the second strand.

9. The nucleic acid adaptor of claim 8, wherein:
the first and the second strands comprise at least 4 phosphorothioates at each end, wherein the phosphorothioates are interspaced by a natural nucleotide species.

10. A kit comprising the nucleic acid adaptor of claim 1.

11. A method for preparing a nucleic acid library, comprising the steps of:
ligating a fully complementary double stranded nucleic acid adaptor to each end of a double stranded nucleic acid target molecule to produce a ligated double stranded adaptor-target-adaptor molecule, wherein the fully complementary double stranded nucleic acid adaptor comprises a plurality of deoxyinosine species positionally located in a spaced relationship from each other on a first adaptor strand that base pair to an A, T, or G nucleotide species on a second adaptor strand;
denaturing the double stranded adaptor-target-adaptor molecule to produce two single stranded adaptor-target-adaptor molecules, wherein the single stranded adaptor-target-adaptor molecules comprise a first region from the first adaptor strand at a first end, a middle region from the nucleic acid target molecule, and a second region from the second adaptor strand at a second end;
annealing a first primer to the second region of a first strand of the single stranded adaptor-target-adaptor molecules;
extending the first primer to produce a second strand complementary to the first strand, wherein the second strand comprises a third region at the second end comprising C nucleotide species base paired to the deoxyinosine species in the first region of the first strand;
annealing a second primer to the third region of second strand; and
extending the second primer to produce a third strand complementary to the second strand, wherein the second and third strands comprise semi-complementary ends resistant to hybridization to each other.

12. The method of claim 11, comprising:
the second strand comprises sequence composition from the first primer at a first end, a middle region from the nucleic acid target molecule, and the third region at a second end.

13. The method of claim 11, comprising:
the third strand comprises second region at a first end, a middle region from the nucleic acid target molecule, and sequence composition from the second primer at a second end.

14. The method of claim 11, comprising:
the second strand and the third strand are each sequencable products.

15. The method of claim 11, further comprising the step of:
clonally amplifying the second strand to produce a population of substantially identical copies; and
sequencing the population of substantially identical copies to produce a sequence composition of the second strand.

16. The method of claim 11, further comprising the step of:
clonally amplifying the third strand to produce a population of substantially identical copies; and
sequencing the population of substantially identical copies to produce a sequence composition of the third strand.

17. The nucleic acid adaptor of claim 11, wherein:
the first adaptor strand comprises a 3′ single base overhang and the second adaptor strand comprises a 5′ phosphate group that preferentially ligates a first end of the the fully complementary double stranded nucleic acid adaptor to each end of the double stranded target nucleic acid molecule.

18. The nucleic acid adaptor of claim 11, wherein:
the deoxyinosine species are spaced 4 or more sequence positions away from each other.

19. The nucleic acid adaptor of claim 11, wherein:
the deoxyinosine species are spaced no closer than 6 sequence positions from an end of the fully complementary double stranded nucleic acid adaptor arranged to ligate to the double stranded target nucleic acid molecule


SEQUENCE LISTING (16 sequences)

SEQ ID NO: 1 - DNA, 30 bp, Artificial Sequence
  Description of Artificial Sequence Synthetic oligonucleotide
cntatcncctgngtgnctnggcngtcgact

SEQ ID NO: 2 - DNA, 29 bp, Artificial Sequence
  Description of Artificial Sequence Synthetic oligonucleotide
gtcgactgccaaggcacacaggggatagg

SEQ ID NO: 3 - DNA, 30 bp, Artificial Sequence
  Description of Artificial Sequence Synthetic primer
cgtatcgcctgggtggctgggcggtcgact

SEQ ID NO: 4 - DNA, 30 bp, Artificial Sequence
  Description of Artificial Sequence Synthetic oligonucleotide
agtcgactgccaaggcacacaggggatagg

SEQ ID NO: 5 - DNA, 30 bp, Artificial Sequence
  Description of Artificial Sequence Synthetic oligonucleotide
cntatnccntgngtgnctnggnagncgact

SEQ ID NO: 6 - DNA, 30 bp, Artificial Sequence
  Description of Artificial Sequence Synthetic oligonucleotide
cgtatgccgtgggtggctggggaggcgact

SEQ ID NO: 7 - DNA, 30 bp, Artificial Sequence
  Description of Artificial Sequence Synthetic oligonucleotide
cntatnccntgngtgncttggnagtcgact

SEQ ID NO: 8 - DNA, 30 bp, Artificial Sequence
  Description of Artificial Sequence Synthetic oligonucleotide
cgtatgccgtgggtggcttgggagtcgact

SEQ ID NO: 9 - DNA, 30 bp, Artificial Sequence; identical to SEQ ID NO: 1
  Description of Artificial Sequence Synthetic oligonucleotide

SEQ ID NO: 10 - DNA, 29 bp, Artificial Sequence
  Description of Artificial Sequence Synthetic oligonucleotide
gtcgactgccaaggcacacaggggatagg

SEQ ID NO: 11 - DNA, 30 bp, Artificial Sequence; identical to SEQ ID NO: 5
  Description of Artificial Sequence Synthetic oligonucleotide

SEQ ID NO: 12 - DNA, 30 bp, Artificial Sequence; identical to SEQ ID NO: 7
  Description of Artificial Sequence Synthetic oligonucleotide

SEQ ID NO: 13 - DNA, 30 bp, Artificial Sequence; identical to SEQ ID NO: 4
  Description of Artificial Sequence Synthetic oligonucleotide

SEQ ID NO: 14 - DNA, 30 bp, Artificial Sequence
  Description of Artificial Sequence Synthetic primer
cctatcccctgtgtgccttggcagtcgact

SEQ ID NO: 15 - DNA, 30 bp, Artificial Sequence
  Description of Artificial Sequence Synthetic oligonucleotide
agtcgaccgcccagccacccaggcgatacg

SEQ ID NO: 16 - DNA, 30 bp, Artificial Sequence
  Description of Artificial Sequence Synthetic oligonucleotide
agtcgaccacccaggcacacaggggatagg